(19) United States
(12) Patent Application Publication
(10) Pub. No.: US 2017/0196944 A1
(43) Pub. Date: Jul. 13, 2017

(54) METHOD TO REDUCE MUSCLE ATROPHY FOLLOWING ORTHOPEDIC SURGERY

(71) Applicant: ROBERT PORTMAN, OCEANPORT, NJ (US)

(72) Inventor: ROBERT PORTMAN, OCEANPORT, NJ (US)

(21) Appl. No.: 14/867,104

(22) Filed: Sep. 28, 2015

(51) Int. Cl. A61K 38/38; A61K 31/7016; A61K 31/7004; A61K 35/20; A61K 31/375; A23L 33/00; A61K 36/48; A23L 33/175; A23L 33/125; A23L 33/19; A23L 33/185; A23L 33/15; A61K 31/198; A61K 9/00

(52) U.S. Cl. CPC A61K 38/38; A61K 31/198; A61K 31/7016; A61K 31/7004; A61K 35/20; A61K 31/375; A61K 9/0095; A61K 36/48; A23L 33/175; A23L 33/125; A23L 33/19; A23L 33/185; A23L 33/15; A23L 33/40; A23V 2002/00

(57) ABSTRACT

A method for reducing muscle atrophy in an individual undergoing orthopedic surgery, the method comprising administering to said individual a mixture of protein and non-protein nutrients for 4-7 days pre-surgery and for 10-21 days post-surgery. The protein dosage is 15-30 g, of which 17-25% is leucine, 23-35% is 1-glutamine, 10-15% is 1-arginine and 1-2% is cysteine. The protein nutrients are derived from a combination of commercially available proteins and the appropriate free amino acid. The non-protein nutrients are a mono or disaccharide in the amount of 15-30%, N-acetyl-cysteine in the amount of 1-15% and the antioxidant Vitamin C.

### FIELD OF THE INVENTION

The present invention relates to a practical cost effective method that reduces the rate of muscle atrophy during the 10-21 day interval post-surgery when muscle atrophy is most assertive because of muscle disuse and because the post-surgical biochemical environment is predisposed to protein degradation. The invention, consisting of a combination of protein, free amino acids, mono or disaccharide, vitamin C and N-acetyl cysteine, works by stimulating multiple nutrient signals that act synergistically to increase protein synthesis while simultaneously inhibiting protein degradation. The invention encourages patient compliance by producing a positive effect with a minimum amount of consumed calories.

### BACKGROUND OF THE INVENTION

Orthopedic surgery involving extremities, knees, hips and shoulders are among the most frequently performed surgeries in the United States. In 2014, almost two million procedures were performed and this number is estimated to triple over the next 20 years as a result of an aging population. Although advanced surgical techniques and a new generation of implant devices have greatly improved outcomes, recovery still represents a challenge to orthopedic surgeons, physical therapists and patients.

The major barrier to functional recovery following orthopedic surgery is muscle atrophy or loss of muscle tissue. On average, patients undergoing a knee or hip replacement two of the most frequently performed procedures, are in the hospital for 1-3 days post-surgery. Depending on the degree of inflammation, trauma and pain, there is usually an additional period of muscle disuse ranging from 7-21 days before the patient can begin physical therapy rehabilitation to restore function in the operative limb.

This interval of muscle disuse has a pronounced effect on the degree of muscle atrophy. Researchers have demonstrated that up to 20% of the muscle mass in the operative limb can be lost within the 10-21 days interval after surgery. Up to 70% of the muscle loss occurs within the first 10 days post-surgery. The accelerated loss of muscle tissue that occurs during this interval has enormous impact on the patient's overall rehabilitation. Since a primary goal of rehabilitation is to restore muscle mass, the greater the muscle loss, the longer the rehabilitation period and the greater cost both to the healthcare system and the patient.

On average, a patient undergoing knee replacement will see a physical therapist 19 times and physical therapy visits can easily exceed 40. A method that would reduce the degree of muscle atrophy that occurs predominately in the 10-21 day period following surgery would significantly reduce healthcare costs since the patient would require less physical therapy in order to return to normal function.

When a load-bearing extremity is not used, it sets in motion a series of metabolic processes that lead to muscle atrophy. Muscle atrophy results in a loss of muscle strength and muscle mass, both of which can seriously impact the speed and quality of recovery. Muscle atrophy following total knee replacement can persist long after wound healing from surgical trauma has been completed. One year after knee replacement surgery a high percentage have significantly reduced muscle strength due to loss of muscle mass. Six to thirteen years after surgery 20% patients still have significant loss of muscle mass in the operative limb.

To better understand what causes muscle atrophy, one must first understand how muscle mass is normally maintained. Maintenance of muscle mass is a dynamic process. New protein is constantly synthesized (anabolism) while old protein is broken down (catabolism).

Normally, this process is in balance. However, this balance can be disrupted both positively and negatively. For example, exercise, particularly resistance exercise, has been shown to increase muscle mass primarily by increasing protein synthesis. When the manufacture of new protein exceeds the breakdown of older protein, there is a net increase in muscle mass. Similarly, there are conditions such as muscle disuse that result in a decrease in muscle mass. In these conditions, the breakdown of protein exceeds the synthesis of protein and muscle mass is lost. The benefits of increased protein synthesis can be neutralized or even overwhelmed if there is a significant increase in protein degradation

Research has shown that both protein synthesis and protein degradation are complex processes involving multiple nutrient signals and metabolic pathways. Nutrient signals can operate independently or synergistically. Two important signaling pathways for increasing protein synthesis are mammalian target of Rapamycin (mTOR) and insulin. The mTOR pathway is activated by essential amino acids, particularly leucine. Leucine has a pronounced effect on protein synthesis.

In addition to leucine and essential amino acids, mTOR can be independently activated by whole protein, such as whey or casein. Studies have shown that combining leucine and whole protein synergistically activates mTOR and protein synthesis.

Another signal for protein synthesis is the hormone insulin. Insulin increases protein synthesis via multiple actions. It independently stimulates mTOR and also increases the transport of amino acids into muscle, thereby facilitating their incorporation into protein. Insulin acts synergistically with whole protein and essential amino acids in increasing protein synthesis. For example adding high glycemic sugars, a stimulus of insulin release, to a meal containing protein or essential amino acids (EAA) increases protein synthesis greater than that seen with protein or EAA given alone.

A fourth nutrient signal for protein synthesis is the amino acid arginine. Arginine improves amino acid transport and independently activates the mTOR pathway.

Finally, a fifth signal is glutamine, the most abundant amino acid found in muscle cells. Glutamine has been shown to independently activate the mTOR pathway.

Similar to protein synthesis, protein degradation is controlled by specific signals. One of the most important is reactive oxygen species (ROS) often called free radicals. Free radicals increase protein degradation. Normally the body has the ability to neutralize reactive oxygen species via glutathione the body's master antioxidant. However when the formation of ROS exceeds glutathione's ability to neutralize them there is an increase in protein degradation.

Glutamine, cysteine and N-acetyl cysteine are three compounds which increase glutathione levels. Raising glutathione levels reduces ROS and inhibits protein degradation. A second signal that inhibits protein degradation is insulin. Thus insulin has a particularly beneficial impact on protein accretion by stimulating protein synthesis and inhibiting protein breakdown.

Although the most effective method to increase protein accretion is to stimulate protein synthesis while simultaneously inhibiting protein degradation, following orthopedic surgery the prevailing biochemical conditions that exist are ideal for decreasing muscle protein. Protein synthesis is inhibited and protein degradation is activated.

Following orthopedic surgery, the mTOR pathway is inhibited, glutamine levels are depleted by up to 40%, glutathione levels are reduced up to 70%, there is a significant increase in oxidative stress as measured by ROS and there is an increase in insulin resistance. Plus there are the practical aspects, immediately following surgery many patients have a decreased appetite which means they don't consume the necessary nutrients to maintain protein status.

Compounding the cellular environment post-surgery are the biochemical conditions in the operative limb prior to surgery. The operative limb pre surgery usually has moderate to severe inflammation measured by an increase in biomarkers of muscle damage and ROS. Inflammation inhibits mTOR signaling which means that protein synthesis in the operative leg is compromised even before the surgery

These multiple factors explain why the rate and severity of muscle atrophy following orthopedic surgery is so much greater than that seen with other conditions that cause muscle atrophy such as extended bed rest, aging and disease states such as cancer and why muscle atrophy following orthopedic surgery requires a qualitatively and quantitatively different approach which is not disclosed in the current art.

FIG. 1 describes the multiple causes of muscle atrophy pre and post-surgery and illustrate why the rate of muscle atrophy following orthopedic surgery is so pronounced.

FIG. 1: The Causes of Muscle Atrophy Pre and Post-Surgery

What is needed in the art is a practical cost effective method that reduces the rate of muscle atrophy during the 10-21 day interval post-surgery when muscle atrophy is most assertive because of muscle disuse, that works by stimulating synergistically multiple signals that activate protein synthesis while simultaneously inhibiting those signals that activate protein degradation and that encourages patient compliance by producing a positive effect with a minimum amount of consumed calories. The method should be, economical, simple to use by all orthopedic patients and does not require professional intervention.

Such an invention would be of enormous benefit both to the patient and to the ultimate cost of health care delivery. By slowing the rate of muscle atrophy following orthopedic surgery, the patient could rehabilitate faster, reduce costs associated with prolonged physical therapy and return more quickly to normal function.

### DESCRIPTION OF THE PRIOR ART

Wolfe et al., U.S. Pat. No. 7,790,688 discloses a composition of matter for increasing muscle mass, strength, and functional performance, comprising: an amino acid component, said amino acid component including: at least 11% percent by mass leucine; and at least one other amino acid, said amino acid selected from a group consisting of: histidine, isoleucine, valine, lysine, methionine, phenylalanine, threonine, arginine, glycine, carnitine, and citrullene; a carbohydrate; and creatine. This prior art does not teach or disclose a method for reducing muscle atrophy caused by orthopedic surgery, the importance of insulin in increasing protein synthesis and decreasing protein degradation or the role ROS play in increasing protein degradation.

Tisdale et al., U.S. Pat. No. 8,329,646 discloses a method of treating muscle loss in an individual, the method comprising: administering to the individual a nutritional product comprising an effective amount of: (i) at least one or a branched chain amino acid (BCAA), a BCAA precursor, a BCAA metabolite, a BCAA-rich protein; or a protein manipulated to enrich the BCAA content, wherein at lest of the BCAA, BCAA precursor, BCAA metabolite, BCAA rich protein, and a protein manipulated to enrich the BCAA content, antagonizes protein catabolism; and (ii) an RNA-dependent protein kinase (PKR) inhibitor, wherein PKR is an interferon-induced serine/threonine protein kinase. This prior art does not teach or disclose a method for reducing muscle atrophy caused by orthopedic surgery in the 10-21 days post-surgery interval when the rate of muscle atrophy is most intense.

Troup et al., U.S. Pat. No. 8,703,725 discloses a composition comprising: leucine, valine in an amount of about 8% to about 10% by weight based on the weight of total amino acids, and at least one essential amino acid in an amount of at least about 15 g in free form per dose, the essential amino acid selected from the group consisting of isoleucine, lysine, methionine, phenylalanine, threonine, tryptophan, histidine, and combinations thereof in free form, the leucine, in free form, being present in an amount of at least 30% to about 95% by weight based on the weight of total amino acids and a ratio of total essential amino acids to total amino acids ranging from about 0.60 to about 0.90. This prior art does not teach or disclose a method for reducing muscle atrophy caused by orthopedic surgery in subjects who are not nutritionally compromised nor does it teach about the role ROS play in increasing protein degradation or the role of intact protein in subjects that are not nutritionally compromised.

Heur et al., U.S. Patent Application 20070015686 discloses a dietary supplement for enhancing GLUT4 protein translocation to the plasma membrane in non-adipose cells, decreasing muscle protein degradation, downregulation of the ATP-dependent ubiquination pathway of muscle catabolism, and decreasing catabolism of muscle cells through reducing the activation of NF-.kappa.beta. The dietary supplement may comprise one or more of high to moderate-glycemic index carbohydrates, dammarane saponins from Gynostemma pentaphyllum, ester-bond containing polyphenols, and creatine and related guanidine compounds. According to various embodiments of the present invention, the dietary supplement may additionally comprise Creatinol-O-phosphate as a source of guanidino compounds. The dietary supplement may also further comprise the antioxidant N-acetyl cysteine (NAC) and the carotenoid, astaxanthin. Furthermore, the dietary supplement may include one or more of a number of branched-chain amino acids and essential amino acids. This prior art does not teach or disclose a method for reducing muscle atrophy caused by orthopedic surgery in the 10-21 days post-surgery interval when the rate of muscle atrophy is most intense.

Sherratt et al., U.S. Pat. No. 6,423,349 discloses a method of promoting recovery from an elective surgical procedure comprising administering to a patient in need thereof, prior to said elective surgical procedure and following said elective surgical procedure, as a daily regimen, a composition in unit dosage form comprising L-glutamine, N-acetyl-cysteine, vitamin A, vitamin C, vitamin E, folic acid, magnesium, selenium, zinc and copper, administered one to two days prior to and two days after surgery. This prior art does not teach or disclose a method for reducing muscle atrophy caused by orthopedic surgery in the 10-21 days post-surgery interval when the rate of muscle atrophy is most intense or the synergistic role of protein, specific amino acids and insulin in increasing protein synthesis while simultaneously decreasing protein degradation. Additionally this invention teaches that the method contain Vitamin E. Because Vitamin E is associated with increased bleeding it is contraindicated before surgery.

Luiking et al., U.S. Pat. No. 8,846,759 discloses a nutritional composition comprising per 100 kcal: (i) at least about 12 g of proteinaceous matter which comprises at least about 80 weight % of whey protein, relative to the total proteinaceous matter, and which comprises at least about 11 weight % of leucine, relative to the total proteinaceous matter, of which at least about 20 weight % is in a free form, relative to the total leucine, (ii) a source of fat and a source of digestible carbohydrates, for use in the prevention or treatment of a disease or condition which involves muscle decline in a mammal, wherein the nutritional composition is administered as 1 to 2 servings daily, each serving comprising between 80 and 200 kcal. This prior art does not teach or disclose a method for reducing muscle atrophy caused by orthopedic surgery in the 10-21 days post-surgery interval when the rate of muscle atrophy is most intense. Additionally this prior art does not teach or disclosed a method for inhibiting protein degradation by decreasing ROS and increasing glutathione levels.

Verlaan et al., U.S. Pat. No. 7,288,570 discloses a composition comprising leucine and proteinaceous matter, wherein leucine is present in an amount of at least about 10 wt. % based on a total dry weight of the composition, in a weight ratio of between about 0.2 and 0.4 leucine to other amino acids present in the proteinaceous matter, and in a weight ratio of greater than about 0.48 leucine to a total amount of branched amino acids. The formulation induces the in vivo production of protein. This prior art does not teach or disclose a method for reducing muscle atrophy caused by orthopedic surgery in the 10-21 days post-surgery interval when the rate of muscle atrophy is most intense. Nor does this prior art teach or discloses a method for simultaneously inhibiting protein degradation by decreasing ROS and increasing glutathione levels.

Ferrando et al., Ser. No. 14/359,213 discloses a method for improving recovery of muscle strength, the method comprising administering to a subject a composition comprising the following concentrations of amino acids in terms of w/w %: about 1 to 2% of histidine, about 9 to 11% of isoleucine, about 35 to 38% of leucine, about 14 to 17% of lysine, about 2 to 4% of methionine, about 5 to 7% of phenylalanine, about 8 to 9% of threonine, about 9 to 11% of valine, about 0 to 0.2% of tryptophan, and about 8 to 11% of arginine, wherein recovery of muscle strength and function of the subject is improved relative to recovery of muscle strength and function in a subject not administered said amino acid composition. Muscle recovery is improved during rehabilitation from a post-surgical trauma including hip surgery trauma and knee surgery. This prior art does not teach or disclose a method for reducing muscle atrophy in the immediate post-surgery interval when the rate of muscle atrophy is most intense. Rehabilitation following orthopedic surgery normally occurs 7-14 days post-surgery. By then most of the muscle atrophy will have taken place. Nor does this art disclose a method for reducing muscle atrophy pre surgery. Nor does this prior art teach or disclose a method for simultaneously and synergistically inhibiting protein degradation by decreasing ROS and increasing glutathione levels.

Lane et al., U.S. Pat. No. 7,572,462 discloses a daily nutritional supplement which comprises: a) about 2000-10,000 IU Vitamin A; b) about 1-10 mg Vitamin B; c) about 0.5-10 mg Vitamin B6; d) about 50-250 mu.g Vitamin B12; e) about 500-10,000 mg Vitamin C; f) about 250-5000 mg L-glutamine; and g) about 30× Arnica Mont. Additionally this patent a nutritional supplement which is specifically tailored to provide nutritional support to an individual during the pre- and post-operative or pre- and post-procedure period (the peri-operative period). This prior art does not teach or disclose a method for reducing muscle atrophy in the immediate post-surgery interval when the rate of muscle atrophy is most intense. Furthermore this prior art does not teach or disclose the use of protein to increase protein synthesis. This prior art also teaches the use of Vitamin E. Because Vitamin E is associated with increased bleeding it is contraindicated before surgery.

It is well known in the art and in science that protein, essential amino acids and carbohydrate can increase protein synthesis alone and in combination. Such compositions have shown to be useful in treating patients with sarcopenia resulting from disease, bed rest and aging. Similarly, the combination of carbohydrate, essential amino acids and protein have been used to successfully stimulate protein synthesis after exercise. In all of these conditions, there is a loss of muscle protein. However, what distinguishes the muscle atrophy that follows orthopedic surgery is the rate and degree.

To place in perspective, as we age we lose muscle mass by approximately 5% per decade. Extended bed rest, a major cause of muscle atrophy only results in a rate of muscle loss of 0.1 to 0.4% per day. Patients with muscle-wasting diseases can lose 20% of their muscle mass within one year or 0.1% per day. Compare these rates to a patient undergoing orthopedic surgery who can lose up to 20% of their muscle mass in the operative limb within 20 days or 1% per day. The greatest percentage of this muscle loss occurs within the first 10 days. FIG. 2 below shows a comparison of muscle loss per day by different muscle wasting conditions.

FIG. 2—Rate of Muscle Atrophy by Condition (Loss/Day)

Although muscle atrophy in the orthopedic patients involves similar mechanisms as that seen in other muscle loss conditions, research shows that unlike other muscle wasting conditions multiple mechanisms are activated to inhibit protein synthesis and activate protein degradation. Timing is critical. Failure to slow muscle atrophy within the critical 10-21 day period after surgery has long-term consequences as evidenced by decreased muscle mass in the operative organ six to 13 years post-surgery.

### OBJECTS OF THE INVENTION

It is an object of the present invention to reduce the rate of muscle atrophy that occurs in the 10-21 day period following orthopedic surgery.

Another object of the present invention is to increase muscle protein synthesis and decrease protein degradation in the 4-7 period prior to surgery.

Another object of the present invention is to increase total amino acid levels in the blood in the 10-21 day period following orthopedic surgery.

Another object of the present invention is to reduce protein degradation in the 10-21 day period following orthopedic surgery.

Another object of the present invention is to reduce the time necessary for full return of muscle strength and function in the 10-21 day period following orthopedic surgery.

Another object of the present invention is to provide a cost-effective palatable method that encourages patient compliance.

Another object of the present invention is to increase plasma levels of leucine, an important amino acid involved in activating protein synthesis.

Another object of the present invention is to reduce oxidative stress that is a normal function of surgical trauma.

Another object of the present invention is to reduce the pain index of patients for four weeks following surgery.

Another object of the present invention is to increase insulin, a hormone involved in activating protein synthesis as well as inhibiting protein degradation.

### SUMMARY OF THE INVENTION

The present invention provides a method for slowing the rate of muscle atrophy following orthopedic surgery by administering a nutrient composition to an individual twice a day 4-7 days prior to surgery and 10-21 following surgery. The invention, consisting of a combination of protein, free amino acids, mono or disaccharide, vitamin C and N-acetyl cysteine, works by stimulating multiple nutrient signals that act synergistically to increase protein synthesis while simultaneously inhibiting protein degradation.

### BRIEF DESCRIPTION OF THE DRAWINGS

FIG. 1 is a diagram showing the multiple biochemical mechanism pre and post-surgery responsible for the increased degree of muscle atrophy following orthopedic surgery.

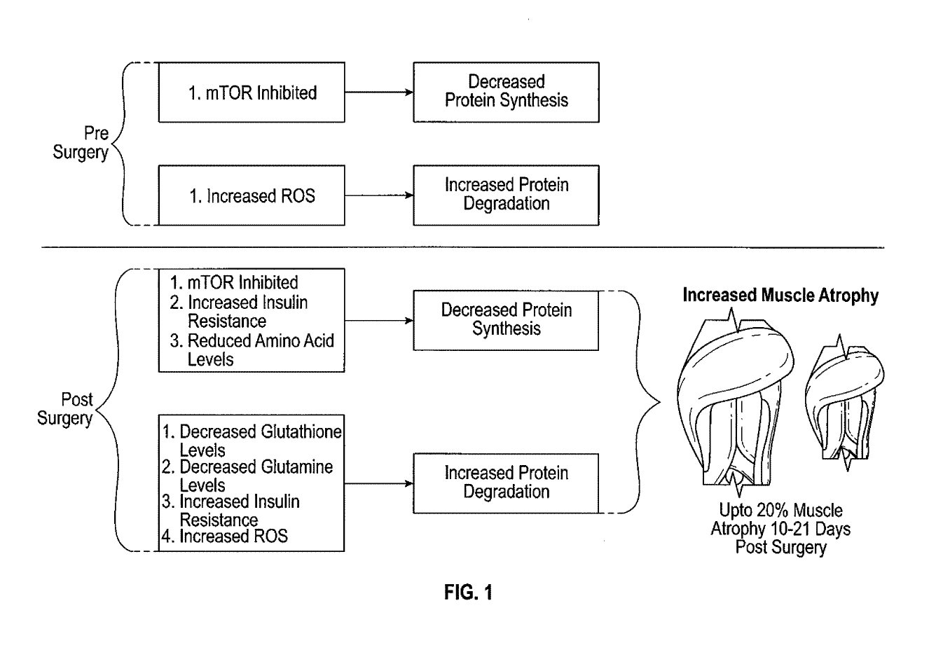

FIG. 2 is chart showing the rate of muscle atrophy in different disease and surgical conditions.

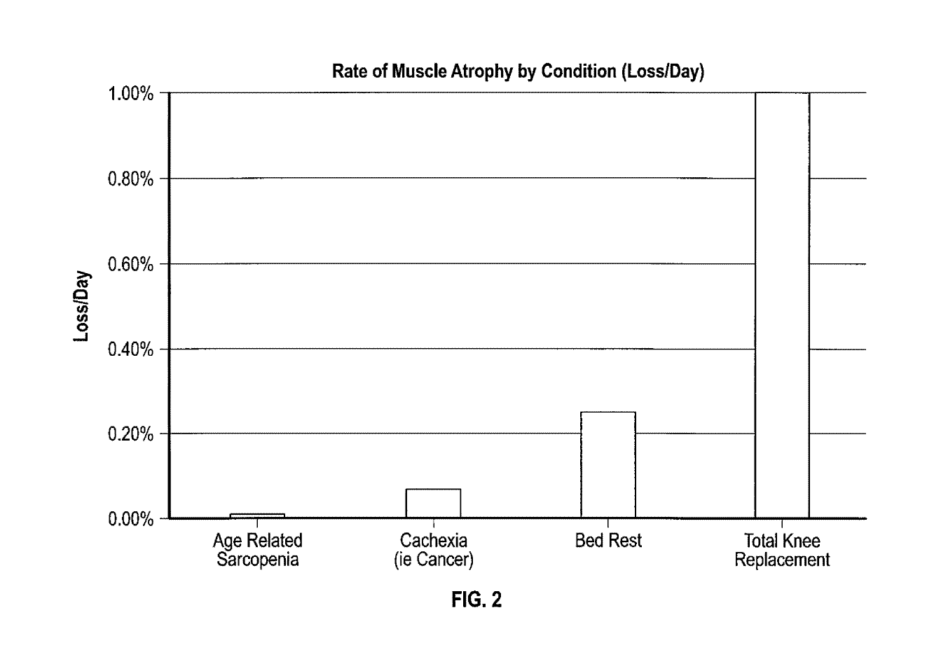

FIG. 3 is a diagram showing how the inventive method decreases muscle atrophy by simultaneously activating multiple biochemical mechanisms to increase protein synthesis and decrease protein degradation before and after surgery.

### DETAILED DESCRIPTION OF THE PREFERRED EMBODIMENT

The inventive method uses a multi-pronged nutritional approach to synergistically stimulate multiple pathways that increase protein synthesis and inhibit multiple pathways that increase protein degradation.

An unexpected result of the invention is that its use of specific nutrients that have multiple effects on protein synthesis. Glutamine not only stimulates mTOR signaling, but also plays a critical role in restoring glutathione levels, thereby inhibiting protein degradation. Arginine also stimulates mTOR signaling and increases amino acid delivery to muscle cells to facilitate protein synthesis.

Similarly, the addition of high glycemic sugars in the form of mono saccharides and/or disaccharides stimulate insulin, a powerful hormone that activates protein synthesis via mTOR signaling, increases protein synthesis independently via an amino acid transport mechanism and also serves as a strong inhibitor of protein degradation.

Another unexpected result of the invention is that consumption of the inventive composition prior to surgery increases muscle protein levels and, at the same time, primes the cells' anabolic machinery since under normal surgical procedure patients do not consume nutrition the night before or the day of surgery. Anabolic priming delays the initiation of surgery-induced muscle atrophy.

Another unexpected result of the invention is that it raises antioxidant levels prior to surgery. One of the major causes of arthritis, the primary reason for knee and hip replacements, is elevated levels of ROS. Increased levels of ROS inhibit mTOR and increase protein degradation. Thus even before surgery the biochemical environment of the operative limb is predisposed toward muscle atrophy.

The preferred embodiment of the inventive method consists of protein, the amino acids leucine, glutamine, arginine and cysteine, a mono or disaccharide, N-acetyl cysteine, vitamin C, a sweetener and coloring in a dry powder that can be mixed with water, juice or skim milk.

Whole protein has been shown to increase protein synthesis by raising blood amino acid levels.

Leucine is an essential amino acid that plays a pivotal role in activating mTOR, the major signaling pathway for protein synthesis. In the preferred embodiment, leucine can be derived from any commercially available protein including whey, pea, casein, rice, soy and egg or consist of the free amino acid.

Glutamine is considered a conditionally essential amino acid. Glutamine is the amino acid found in the largest concentration in the muscle. Glutamine has multiple roles in stimulating protein synthesis and inhibiting protein degradation. Glutamine activates mTOR by controlling leucine transport into the muscle. Glutamine is also one of the amino acids comprising glutathione, the body's master antioxidant. Glutathione, by neutralizing reactive oxygen species, prevents protein degradation since ROS are an important signal for activating protein degradation. In the preferred embodiment, glutamine can be derived from any commercially available protein. In the preferred embodiment, leucine can be derived from any commercially available protein including whey, pea, casein, rice, soy and egg or consist of the free amino acid.

The amino acid cysteine is also one of the three amino acids, along with glycine and glutamine that comprises glutathione. Cysteine consumption is the rate-limiting step for glutathione synthesis. In the preferred embodiment, cysteine can be derived from cysteine-enriched proteins such as lactalbumin or egg protein.

Arginine is an amino acid that independently activates the mTOR signaling pathway and also plays an important role in increasing the transport of key nutrients, such as amino acids, into the muscle cells. In the preferred embodiment, arginine can be derived from any commercially available protein including whey, pea, casein, rice soy and egg or consist of the free amino acid.

N-acetyl cysteine is a sulphur-containing compound that contains cysteine, a critical substrate for the synthesis of glutathione.

Vitamin C is an antioxidant that can be used safely with patients undergoing a surgical procedure. By reducing ROS, vitamin C neutralizes a key element involved in activating protein degradation. Unlike the antioxidant Vitamin E, Vitamin C is not contraindicated prior to surgery.

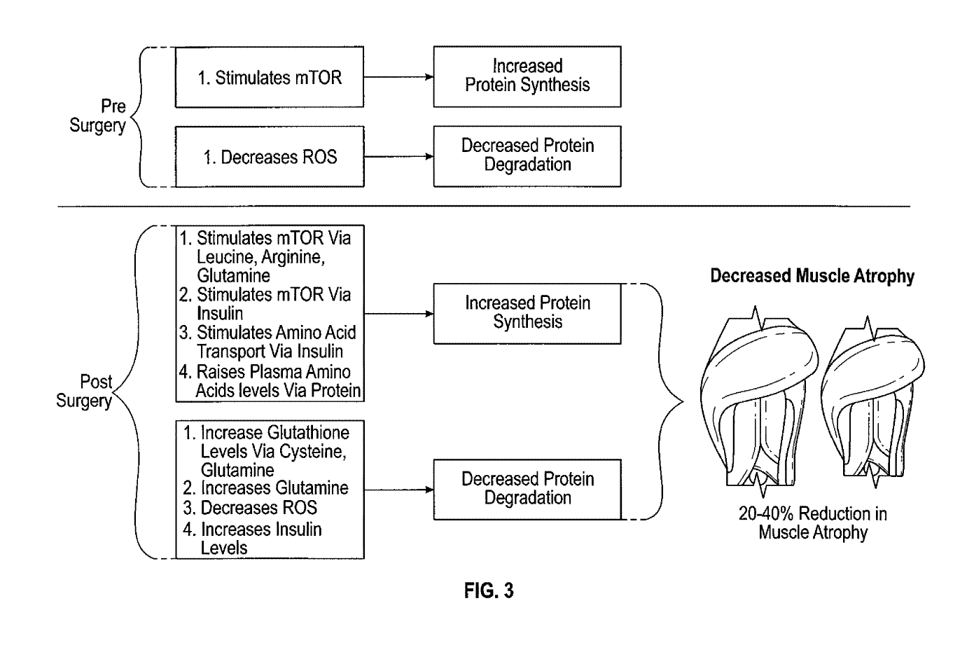

FIG. 3 shows how the inventive methods works by simultaneously activating multiple mechanisms to increase protein synthesis and decrease protein degradation pre and post-surgery.

FIG. 3—How the Inventive Method Works

The preferred embodiment of the inventive method contains a high glycemic sugar in the form of a mono or disaccharide such as glucose, glucose polymers, dextrose, maltose, maltodextrin, sucrose, high fructose corn syrup, beet sugar, or cane sugar. High glycemic sugars are more effective in stimulating insulin, a critical hormone that is responsible for activating protein synthesis thru multiple pathways and for inhibiting protein degradation.

The preferred embodiment includes a flavor component for imparting a characteristic taste and a sweetener to improve palatability.

The range of intact protein ranges from 15 to 30 grams per serving. The intact protein can be selected from a combination of commercially available proteins including whey, pea, casein, lactalbumin, rice and soy.

The inventive method requires a specific amount of the amino acids leucine, arginine, glutamine and cysteine as shown in the table below. Since all intact proteins contain these amino acids in varying amounts, the combination of proteins selected determines the amount of free amino acids that must be added to the inventive composition to achieve the required levels. The free amino acids added will vary based on the different types of intact protein used in the inventive composition. As an example Table 1 below shows the different levels per gram of arginine, glutamine and leucine, in six different commercially available proteins.

TABLE 1

| | | | | Whey enriched | | |
|---|---|---|---|---|---|---|
| | | | | with | | |
| | Whey | Pea | Casein | Lactalbumin | Soy | Egg |
| Arginine | 0.02 | 0.09 | 0.03 | 0.03 | 0.08 | 0.05 |
| Glutamine | 0.14 | 0.17 | 0.18 | 0.13 | 0.19 | 0.12 |
| Leucine | 0.09 | 0.08 | 0.08 | 0.08 | 0.08 | 0.07 |

Table 2 below illustrates the range of leucine, glutamine, arginine and cysteine as a percentage of total protein at three different levels, 15, 20 and 30 grams of protein. The range of leucine is 17% to 25% of total protein. The range of glutamine is 23% to 35% of total protein. The range of arginine is 10% to 15% of total protein. The range of cysteine is 1 to 2% of total protein.

TABLE 2

| | LOW | | PREFERRED | | HIGH | |
|---|---|---|---|---|---|---|
| | PROTEIN | | PROTEIN | | PROTEIN | |
| | LEVEL | | LEVEL | | LEVEL | |
| | (15 grams) | | (20 grams) | | (30 grams) | |
| | | % | | % | | % |
| | Grams | Protein | Grams | Protein | Grams | Protein |
| Leucine | 2.5 | 17% | 5 | 25% | 7.5 | 25% |
| Glutamine | 3.5 | 23% | 7 | 35% | 10.5 | 35% |
| Arginine | 1.5 | 10% | 3 | 15% | 4.5 | 15% |
| Cysteine | 0.15 | 1% | 0.3 | 2% | 0.45 | 2% |

### Example of Preferred Embodiment

| | | % Dry |
|---|---|---|
| Ingredient | g | Weight |
| Whey Protein | 9.0 | 26% |
| Whey enriched with lactalbumin | 7.0 | 20% |
| Pea Protein | 4.0 | 12% |
| Free Leucine | 3.0 | 9% |
| Free Glutamine | 4.0 | 12% |
| Free Arginine | 2.0 | 6% |
| Cane Sugar | 4.0 | 12% |
| N-acetyl cysteine | 0.5 | 1% |
| Vitamin C | 0.2 | 1% |
| Sweetener | 0.1 | 0% |
| Flavor | 0.8 | 2% |
| | 34.6 | 100% |

### Example of Second Embodiment

| | | % Dry |
|---|---|---|
| Ingredient | g | Weight |
| Casein | 4.0 | 16% |
| Whey Protein | 3.0 | 12% |
| Whey enriched with lactalbumin | 5.0 | 20% |
| Pea Protein | 3.0 | 12% |
| Free Leucine | 1.5 | 6% |
| Free Glutamine | 1.3 | 5% |
| Free Arginine | 1.0 | 4% |
| Cane sugar | 4.0 | 16% |
| N-acetyl cysteine | 0.5 | 2% |
| Vitamin C | 0.2 | 1% |
| Flavor | 0.8 | 3% |
| Sweetener | 0.1 | 0% |
| TOTAL | 24.4 | 100% |

### Example of Third Embodiment

| | | % Dry |
|---|---|---|
| Ingredient | g | Weight |
| Casein | 5.0 | 10% |
| Whey Protein | 10.0 | 20% |
| Whey enriched with lactalbumin | 10.0 | 20% |
| Pea Protein | 5.0 | 10% |
| Free Leucine | 5.0 | 10% |
| Free Glutamine | 6.2 | 12% |
| Free Arginine | 3.2 | 6% |
| Cane sugar | 4.0 | 8% |
| N-acetyl cysteine | 0.5 | 1% |
| Vitamin C | 0.2 | 0% |
| Flavor | 0.8 | 2% |
| Sweetener | 0.1 | 0% |
| TOTAL | 50.0 | 100% |

### Example of Fourth Embodiment

| | | % Dry |
|---|---|---|
| | g | Weight |
| Whey Protein | 8 | 23% |
| Pea Protein | 4 | 11% |
| Soy Protein | 4 | 11% |
| Egg Protein | 4 | 11% |
| Free Leucine | 3.4 | 10% |
| Free Glutamine | 4.1 | 12% |
| Free Arginine | 2.1 | 6% |
| Cane Sugar | 4 | 11% |
| N-acetyl cysteine | 0.5 | 1% |
| Vitamin C | 0.2 | 1% |
| Flavor | 0.8 | 2% |
| Sweetener | 0.1 | 0% |
| TOTAL | 35.2 | 100% |

### Example of Fifth Embodiment

| | | % Dry |
|---|---|---|
| | g | Weight |
| Casein | 4.0 | 11% |
| Whey Protein | 5.0 | 13% |
| Pea Protein | 4.0 | 11% |
| Soy Protein | 3.0 | 8% |
| Egg Protein | 4.0 | 11% |
| Cane sugar | 6.0 | 16% |
| N-acetyl cysteine | 1.0 | 3% |
| Free Glutamine | 3.9 | 10% |
| Free Leucine | 3.3 | 9% |
| Free Arginine | 2.0 | 5% |
| Vitamin C | 0.2 | 1% |
| Flavor | 0.8 | 2% |
| Sweetener | 0.1 | 0% |
| TOTAL | 37.3 | 100% |

Administration of the inventive method to a group of total knee replacement patients four to seven days prior to surgery and 10 to 14 days post-surgery would produce the following results when compared to a similar group that did not receive the inventive method:

1. A 20-40% drop in muscle atrophy within 14 days after surgery.

2. An increase in total plasma amino acid levels within 14 days after surgery, thereby ensuring that protein synthesis continues at optimum levels.

3. An increase in plasma leucine within 14 days after surgery, thereby ensuring that protein synthesis continues at optimum levels.

4. An increase in plasma glutamine levels within 14 days after surgery, thereby increasing glutathione levels.

5. A decrease in ROS within 14 days after surgery thereby decreasing protein degradation.

6. An increase in muscle strength within 14 days after surgery.

7. A decrease in pain parameters within 21 days after surgery.

8. An increase in normal function within 21 days after surgery.

### ADVANTAGES OF THE PRESENT INVENTION

An advantage of the present invention is to reduce the rate of muscle atrophy that occurs in the 10-21 day period following orthopedic surgery by 20-40%.

Another advantage of the present invention is to increase muscle protein synthesis in the 4-7 period prior to surgery.

Another advantage of the present invention is to increase total amino acid levels in the blood in the 10-21 day period following orthopedic surgery.

Another advantage of the present invention is to reduce protein degradation in the 10-21 day period following orthopedic surgery.

Another advantage of the present invention is to reduce the time necessary for full return of muscle strength and function in the 10-21 day period following orthopedic surgery.

Another advantage of the present invention is to provide a cost-effective palatable method that encourages patient compliance.

Another advantage of the present invention is to increase plasma levels of leucine, an important amino acid involved in activating protein synthesis.

Another advantage of the present invention is to reduce oxidative stress that is a normal consequence of surgical trauma.

Another advantage of the present invention is to reduce the pain index of patients for four weeks following surgery.

Another advantage of the present invention is to increase insulin, a hormone involved in activating protein synthesis as well as inhibiting protein degradation.

Another advantage of the present invention is to reduce muscle atrophy in an individual who requires partial immobilization due to injury.

Another advantage of the present invention is to simultaneously and synergistically activate multiple anabolic and multiple anti-catabolic pathway in an individual who requires partial immobilization due to injury

A latitude of modification, change and substitution is intended in the foregoing disclosure, and in some instances, some features of the invention will be employed without a corresponding use of other features. Accordingly, it is appropriate that the appended claims be construed broadly and in a manner consistent with the spirit and scope of the invention herein.

### REFERENCES

Anthony, et al., “Leucine Stimulates Translation Initiation in Skeletal Muscle of Post absorptive Rats via a Rapamycin-Sensitive Pathway”, J Nutr, 2000, pp. 2413-2419.
Bar-Peled et al., “Regulation of mTORC1 by amino acids” (2014) Trends Cell Biol 1-7.
Del Salvio et al., “Preoperative nutrition status and outcome of elective total hip replacement” (1996) Clin Orthop Relat Res 326:153-161.
Douglas-Paddon et al., “Dietary protein recommendations and the prevention of sarcopenia” (2012) Curr Opin Clin Nutr Metab Care 12(1):86-90.
Dreyer et al., “Leucine-enriched essential amino acids and carbohydrate ingestion following resistance exercise enhances mTOR signaling and protein synthesis in human muscle” (2007) Am J Physiol Endocrinol Metab 294:E392-E400.
English et al., “Protecting muscle mass and function in older adults during bed rest” (2010) Curr Opin Clin Nutr Metab Care 13.1:34-39.
Fujita et al., “Nutrient signaling in the regulation of human muscle protein synthesis” (2007) J Physiol 582.2:813-823.
Jackman et al., “The molecular basis of skeletal muscle atrophy” (2004) Am J Physiol Cell Physiol 287:C834-C843.
Katsanos, et al., “A High Proportion of Leucine is Required for Optimal Stimulation of the Rate of Muscle Protein Synthesis by Essential Amino Acids in the Elderly”, Am J Physiol Endocrinol Metab, 2006, pp. E381-E387.
Kimball et al., “Exercise effects on muscle insulin signaling and action. Invited Review: Role of insulin in translational control of protein synthesis in skeletal muscle by amino acids or exercise” (2002) J Appl Physiol 93:1168-1180.
Koopman et al., “Combined ingestion of protein and free leucine with carbohydrate increases postexercise muscle protein synthesis in vivo in male subjects” (2004) Am J Physiol Endocrinol Metab 288: E645-E653).
Marzani, et al., “Antioxidant Supplementation Restores Defective Leucine Stimulation of Protein Synthesis in Skeletal Muscle from Old Rats”, J Nutr, 2008, pp. 2205-2211.
Medved et al., “N-acetylcysteine enhances muscle cysteine and glutathione availability and attenuates fatigue during prolonged exercise in endurance-trained individuals” (2004) J Appl Physiol 97:1477-1485.
Oatis et al., “Variations in delivery and exercise content of physical therapy rehabilitation following total knee replacement surgery: a cross-sectional observation study” (2014) Int J Phys Med Rehabil S5:002.
Paddon-Jones et al., “Dietary protein recommendations and the prevention of sarcopenia” Paddon-Jones, et al., “Amino Acid Ingestion Improves Muscle Protein Synthesis in the Young and Elderly”, Am J Physiol Endocrinol Metab, 2003, pp. E321-E328.
Patel et al., “Cellular stresses profoundly inhibit protein synthesis and modulate the states of phosphorylation or multiple translation factors” (2002) Eur J Biochem 269:3076-3085.
Powers et al., “Oxidative stress and disuse muscle atrophy: cause or consequence?” (2012) Curr Opin Clin Nutr Metab Care 15(3):240-245.
Ranga et al., “Isoleucine and leucine independently regulate mTOR signaling and protein synthesis in MAC-T cells and bovine mammary tissue slices” (2012) J Nutri 484-491.
Rieu et al., “Leucine supplementation improves muscle protein synthesis in elderly men independently of hyperaminoacidaemia” (2006) J Physiol 575:1:305-315.
Rieu, et al., “Increased Availability of Leucine with Leucine-Rich Whey Proteins Improves Postprandial Muscle Protein Synthesis in Aging Rats”, Nutrition, 2007, pp. 323-331.
Rieu, et al., “Leucine Supplementation Improves Muscle Protein Synthesis in Elderly Men Independently of Hyperaminoacidaemia”, J Physiol, 2006, pp. 305-315.
Wolfe, Robert R., “Protein Supplements and Exercise”, Am J Clin Nutr., 2000, pp. 551S-557S.

### U.S. PATENT DOCUMENTS

| | | |
|---|---|---|
| 7,790,688 | July 2002 | Wolfe et al. |
| 8,329,646 | December 2012 | Tisdale et al. |
| 8,703,725 | April 2014 | Troup et al. |
| 6,423,349 | July 2002 | Sherratt et al. |
| 8,846,759 | September 2014 | Luiking et al |
| 7,288,570 | June 2004 | Verlaan et al. |
| 20140343112 | November 2014 | Ferrando et al. |
| 20070015686 | January 2007 | Heur et al. |

What is claimed is:

1. A method for reducing muscle atrophy in an individual undergoing orthopedic surgery in which the operative limb undergoes any degree of muscle disuse post-surgery, the method comprising the step of administering to the individual a composition of proteins, amino acids, and non-protein nutrients 4-7 days pre surgery and 10-21 days post-surgery for simultaneously and synergistically activating multiple anabolic pathways and multiple anti-catabolic pathways in the operative limb to increase protein synthesis while inhibiting protein degradation.

2. The method of claim 1, wherein said amino acids include leucine, glutamine, arginine, and cysteine, and wherein said non-protein nutrients include a mono saccharide or a disaccharide, and N-acetyl cysteine.

3. The method of claim 1, wherein said amino acids include leucine in the amount of 2.5 to 7.5 grams, glutamine in the amount of 3.5 to 10.5 grams, arginine in the amount of 1.5 to 4.5 grams, and cysteine in the amount of 0.15 to 0.45 grams, and wherein said non-protein nutrients include a mono saccharide or a disaccharide, and N-acetyl cysteine.

4. The method of claim 1, wherein the composition of protein, amino acid and non-protein nutrients optimally activates the mTOR, insulin-dependent and glutathione pathways and inhibits the formation of ROS.

5. The method of claim 1, wherein the schedule for administering the dose of protein, amino acid and non-protein nutrients is 1-3 times per day.

6. The method of claim 1, wherein the dosage unit of the protein nutrients is 15-30 grams.

7. The method of claim 1, wherein the amino acid composition contains 17-25% 1-leucine by weight based on the total dry weight of the protein nutrients.

8. The method of claim 7, wherein the composition of leucine will optimally activate the mTOR pathway.

9. The method of claim 7, wherein the amount 1-leucine is derived from a combination of proteins including whey, pea, casein, lactalbumin, rice, soy, and egg, and free leucine.

10. The method of claim 1, wherein the amino acid composition contains 23-35% glutamine by weight based on the total dry weight of the protein nutrients.

11. The method of claim 10, wherein the glutamine composition will restore glutathione levels and optimally activate the mTOR pathway.

12. The method of claim 10, wherein the amount 1-glutamine is derived from a combination of proteins including whey, pea, casein, lactalbumin, rice, soy and egg, and free glutamine.

13. The method of claim 1, wherein the amino acid contains 10-15% 1-arginine by weight based on the total dry weight of the protein nutrients.

14. The method of claim 13, wherein the arginine composition will optimally activate the mTOR pathway.

15. The method of claim 13, wherein the amount of 1-arginine is derived from a combination of proteins including whey, pea, casein, lactalbumin, rice, soy and egg, and free arginine.

16. The method of claim 1, wherein the amino acid composition of the protein nutrients contains 1-2% 1-cysteine by weight based on the total dry weight of the protein nutrients.

17. The method of claim 16, wherein the amount 1-cysteine is derived from a combination of proteins including whey, pea, casein, lactalbumin, rice, soy and egg.

18. The method of claim 1, wherein the non-protein nutrient is a monosaccharide or disaccharide in the amount of 15-30% by weight based on the total dry weight of the composition.

19. The method of claim 18, wherein the monosaccharide or disaccharide activates insulin dependent pathways.

20. The method of claim 1, wherein the non-protein nutrient is N-acetyl cysteine in the amount of 3-15% by weight based on the total dry weight of the composition.

21. The method of claim 1, wherein vitamin C is present in 100-500% of the RDA.

22. The method of claim 1, wherein the method decreases the pain index in a patient within 4 weeks after surgery.

23. The method of claim 1, wherein the method increases physical function within 4 weeks after surgery.

24. The method of claim 1, wherein the method increases total plasma protein levels within 4 weeks after surgery.

25. The method of claim 1, wherein the method increases the blood amino acid level of leucine by at least 10% within 2 weeks after surgery.

26. The method of claim 1, wherein the method increases the blood amino acid level of glutamine by at least 10% within 2 weeks after surgery.

27. The method of claim 1, wherein the method increases the blood amino acid level of arginine by at least 10% within 2 weeks after surgery.

28. The method of claim 1, wherein the method increases the levels of the active form of glutathione by at least 10% in the body, within 2 weeks after surgery.

29. The method of claim 1, wherein the method decreases reactive oxygen species by at least 10% in the body, within 2 weeks after surgery.

30. The method of claim 1, wherein the form of the composition is a pharmaceutical composition, a food product or a dietary supplement.

31. The method of claim 1, wherein the composition is in the form of a dry powder that is reconstituted with water, juice or skim milk.

32. The method of claim 1, wherein the composition is in the form of a ready to drink beverage, in the form of a bar, or in the form of a gel.

33. The method of claim 1, wherein the composition has a total caloric composition of 90-250 calories.

34. The method of claim 1, wherein the composition has a total weight of 20 to 50 grams.

35. A method for reducing muscle atrophy in an individual who requires partial immobilization due to injury, the method comprising the step of administering to the individual a composition of proteins, amino acids, and non-protein nutrients 10-21 days post-injury for simultaneously and synergistically activating multiple anabolic and multiple anti-catabolic pathways in the injured limb to increase protein synthesis while inhibiting protein degradation.

36. The method of claim 35, wherein said amino acids include leucine, glutamine, arginine, and cysteine, and wherein said non-protein nutrients include a monosaccharide or a disaccharide, and N-acetyl cysteine.

37. The method of claim 35, wherein said amino acids include leucine in the amount of 2.5 to 7.5 grams, glutamine in the amount of 3.5 to 10.5 grams, arginine in the amount of 1.5 to 4.5 grams, and cysteine in the amount of 0.15 to 0.45 grams, and wherein said nutrients are a monosaccharide or a disaccharide, and N-acetyl cysteine.

38. The method of claim 35, wherein the composition of protein, amino acid and non-protein nutrients optimally activates the mTOR, insulin-dependent and glutathione pathways and inhibits the formation of ROS.

39. The method of claim 35, wherein the dosage schedule for administering the dose of protein nutrients is 1-3 times per day.

40. The method of claim 35, wherein the amino acid composition contains 17-25% 1-leucine by weight based on the total dry weight of the protein nutrients.

41. The method of claim 40, wherein the amount of 1-leucine is derived from a combination of proteins including whey, pea, casein, lactalbumin, rice, soy and egg, and free leucine.

42. The method of claim 35, wherein the amino acid composition s contains 23-35% 1-glutamine by weight based on the total dry weight of the protein nutrients.

43. The method of claim 42, wherein the amount of 1-glutamine is derived from a combination of proteins including whey, pea, casein, lactalbumin, rice, soy and egg and free glutamine.

44. The method of claim 35, wherein the amino acid composition contains 10-15% 1-arginine by weight based on the total dry weight of the protein nutrients.

45. The method of claim 44, wherein the amount 1-arginine is derived from a combination of proteins including whey, pea, casein, lactalbumin, rice, soy and egg, and free arginine.

46. The method of claim 36, wherein the amino acid composition contains 1-2% 1-cysteine by weight based on the total dry weight of the protein nutrients.

47. The method of claim 46, wherein the amount 1-cysteine can be derived from any combination of proteins including whey, pea, casein, lactalbumin, rice, soy and egg.

48. The method of claim 36, wherein the nutrients include a monosaccharide or disaccharide in the amount of 10-20% by weight based on the total dry weight of the composition.

49. The method of claim 36, wherein the nutrient is N-acetyl cysteine is in the amount of 1-15% by weight based on the total dry weight of the composition.

50. The method of claim 36, wherein vitamin C is present in 100-500% of the RDA.

51. The method of claim 36, wherein the form of the composition is a pharmaceutical composition, a food product or a dietary supplement.

52. The method of claim 36, wherein the composition is in the form of a dry powder that is reconstituted with water, juice or skim milk.

53. The method of claim 36, wherein the composition is in the form of a ready to drink beverage, in the form of a bar, or in the form of a gel.

54. The method of claim 36, wherein the composition has a total caloric composition of 90-250 calories.

55. The method of claim 36, wherein the composition has a total weight of 20-50 grams.

56. A method for reducing disuse atrophy in an individual who is confined to a bed for an extended period or who has age related sarcopenia, the method comprising the step of administering to the individual a composition of proteins, amino acids, and non protein nutrients to simultaneously and synergistically activate multiple anabolic pathways to increase muscle protein synthesis and multiple anti-catabolic pathways to inhibit protein degradation.

57. The method of claim 56, wherein said amino acids include leucine, glutamine, arginine, and cysteine, and wherein said non-protein nutrients include a monosaccharide or a disaccharide, and N-acetyl cysteine.

58. The method of claim 56, wherein said amino acids include leucine in the amount of 2.5 to 7.5 grams, glutamine in the amount of 3.5 to 10.5 grams, arginine in the amount of 1.5 to 4.5 grams, and cysteine in the amount of 0.15 to 0.45 grams, and wherein said nutrients are a monosaccharide or a disaccharide, and N-acetyl cysteine.

59. The method of claim 56, wherein the composition of protein, amino acid and non-protein nutrients optimally activates the mTOR, insulin-dependent and glutathione pathways and inhibits the formation of ROS.

60. The method of claim 56, wherein the dosage schedule for administering the dose of protein, amino acids and non-protein nutrients is 1-3 times per day.

61. The method of claim 56, wherein the amino acid composition contains 17-25% 1-leucine by weight based on the total dry weight of the protein nutrients.

62. The method of claim 61, wherein the amount of 1-leucine is derived from a combination of proteins including whey, pea, casein, lactalbumin, rice, soy and egg and free leucine.

63. The method of claim 56, wherein the amino acid composition s contains 23-35% 1-glutamine by weight based on the total dry weight of the protein nutrients.

64. The method of claim 63, wherein the amount of 1-glutamine is derived from a combination of proteins including whey, pea, casein, lactalbumin, rice, soy and egg and free glutamine.

65. The method of claim 59, wherein the amino acid composition contains 10-15% 1-arginine by weight based on the total dry weight of the protein nutrients.

66. The method of claim 65, wherein the amount 1-arginine is derived from a combination of proteins including whey, pea, casein, lactalbumin, rice, soy and egg and free arginine.

67. The method of claim 56, wherein the amino acid composition contains 1-2% 1-cysteine by weight based on the total dry weight of the protein nutrients.

68. The method of claim 67, wherein the amount 1-cysteine can be derived from any combination of proteins including whey, pea, casein, lactalbumin, rice, soy and egg

69. The method of claim 56, wherein the nutrients include a monosaccharide or disaccharide in the amount of 10-20% by weight based on the total dry weight of the composition.

70. The method of claim 56, wherein the nutrient is N-acetyl cysteine is in the amount of 1-15% by weight based on the total dry weight of the composition.

71. The method of claim 56, wherein vitamin C is present in 100-500% of the RDA.

72. The method of claim 56, wherein the form of the composition is a pharmaceutical composition, a food product or a dietary supplement.

73. The method of claim 56, wherein the composition is in the form of a dry powder that is reconstituted with water, juice or skim milk.

74. The method of claim 56, wherein the composition is in the form of a ready to drink beverage, in the form of a bar, or in the form of a gel.

75. The method of claim 56, wherein the composition has a total caloric composition of 90-250 calories.

76. The method of claim 56, wherein the composition has a total weight of 20-50 grams.
